(19) United States
(12) Patent Application Publication
(10) Pub. No.: US 2005/0100608 A1
(43) Pub. Date: May 12, 2005

(54) Testosterone oral dosage formulations and associated methods

(75) Inventor: Charles Ebert, Salt Lake City, UT (US)

Correspondence Address: THORPE NORTH & WESTERN, P.O. BOX 1219, SANDY, UT, 84091-1219, US

(73) Assignee: Watson Pharmaceuticals, Inc.

(21) Appl. No.: 10/784,905

(22) Filed: Feb. 23, 2004

Related U.S. Application Data
(60) Provisional application No. 60/448,488, filed on Feb. 21, 2003.

(51) Int. Cl. A61K 31/56; A61K 31/57; A61K 9/14

(52) U.S. Cl. 424/486; 514/177

(57) ABSTRACT

Solid oral dosage forms of testosterone and methods for the preparation thereof are disclosed and described. The solid oral dosage form may include a therapeutically effective amount of testosterone in a substantially solid polyethylene glycol carrier. Such a form has been found to alleviate many of the undesirable consequences of undergoing testosterone therapy, such as the pain of injections and problems with patient noncompliance.

### PRIORITY DATA

This application claims priority to U.S. Provisional Patent Application Ser. No. 06/448,488, filed on Feb. 21, 2003, which is incorporated herein by reference.

### FIELD OF THE INVENTION

The present invention relates to oral testosterone dosage formulations and to methods for the making and use thereof. Accordingly, this invention involves the fields of chemistry, pharmaceutical science, and medicine.

### BACKGROUND OF THE INVENTION

Androgens are typically defined to include any natural or synthetic steroid hormone that stimulates or controls masculine traits in vertebrates, particularly mammals. One naturally occurring androgen is testosterone, a steroid hormone that has numerous physiological effects in humans, both male and female. In males, for example, testosterone and its related metabolites play a major role in the stimulation, subsequent development, and the ongoing maintenance of the male reproductive system. Similarly, testosterone plays a critical role in the generation and maintenance of male secondary sexual characteristics. Testosterone also plays a role in the development of the female reproductive system, though to a much lesser extent than what is seen in developing males.

Testosterone is often utilized in the medical arts as a treatment for numerous human ailments and disorders. It is used for replacement therapy in men who experience climacteric symptoms associated with male menopause, as well as those individuals that have disorders of the reproductive system, such as hypogonadism. It has also been shown to be beneficial for the treatment of hypopituitarism and Addison's disease. Also, due to its effect of increasing libido in both males and females, testosterone has been successfully used to treat deficiencies in sexual desire. This steroid hormone may also be useful in treating pituitary dwarfism due to its stimulatory effects on the pituitary gland causing accelerated growth. Additional known uses include the treatment of menopause, osteoporosis, endometriosis, vasomotor instability in postmenopausal women, dysmenorrhea, and may reduce some of the pain associated with breast cancer.

On means of administering drugs in the medical arts is by a liquid dosage form. Such liquid dosage forms may be given intravenously, intramuscularly, or by mouth in the form of a drinkable liquid, a spray, or a liquid filled capsule to name a few. Liquids can be problematic for many reasons. Drugs that are given intramuscularly or intravenously usually must be given in a medical environment by someone trained to administer the liquid. Many patients may hesitate to receive the drug in this manner due to pain and inconvenience, which often results in low patient compliance. There is also an increased chance of infections due to the physical penetration of the recipient's skin. Drugs in drinkable liquids, either in solution or emulsion, require mixing that tends to be messy. Additionally, liquid suspension or solutions may also require refrigeration. There is always a likelihood that the liquid will spill, and that the patient may not receive the full intended dose of the drug. Also, it is usually difficult to mask unpleasant drug tastes in liquid solutions and sprays. Liquid capsules alleviate many of these problems, however, liquids may cause difficulties in the manufacturing process that are not seen with solid forms of drugs.

Solid oral dosage forms may represent an improvement for certain drugs over many of the problematic aspects of liquid forms. These forms of administration may include tablets, capsules, caplets, powders, pellets, granules, etc. The drug can be provided in a small, easy to administer form, that greatly increases the likelihood that the patient will receive all of the intended drug administration. Also, the solid dosage form may be coated to reduce unpleasant tastes associated with a given drug. One example of a solid dosage form that has been used for administration of progesterone and estrogen in U.S. Pat. No. 6,544,553, which hereby incorporated by reference.

At present, the most prevalent mode of testosterone administration seems to be either via a liquid injection, or transdermal administration. However, as a result of the significant advantages mentioned, a solid dosage form containing testosterone continues to be sought.

### SUMMARY OF THE INVENTION

Applicants have discovered an effective solid oral dosage formulation of testosterone in a substantially solid polyethylene glycol carrier. This oral dosage form alleviates many of the undesirable consequences of undergoing testosterone therapy, such as the pain of injections and problems with patient noncompliance.

Accordingly, in one aspect, the present invention provides an oral dosage testosterone formulation, for administration to a subject, comprising testosterone dispersed in a substantially solid polyethylene glycol carrier. In one aspect, the present invention provides an amount of testosterone in the substantially solid polyethylene glycol carrier of from about 5 mg to about 15 mg. In another aspect, the amount of testosterone in the solid carrier may be about 10 mg.

The present invention provides that the carrier of the oral dosage formulation be substantially solid polyethylene glycol. In one aspect of the present invention, the polyethylene glycol used may have an average molecular weight of from about 100 to about 20,000 or a mixture thereof. In another aspect, the polyethylene glycol carrier may have an average molecular weight of from about 1,000 to about 10,000 or a mixture thereof. Furthermore, in some aspects, the oral dosage formulation may include an amount of substantially solid polyethylene glycol of from about 30% w/w to about 80% w/w of the oral dosage formulation. In another aspect, the amount may be from about 50% w/w to about 80% w/w of the oral dosage formulation. In a further aspect, the amount may be from about 60% w/w to about 80% w/w. In yet another aspect, the amount may be 70% w/w of the oral dosage formulation.

In addition to the formulations presented herein, the present invention additionally encompasses methods of making and using such formulations. In one aspect, the present invention provides a method of administering testosterone to a subject, either for replacement therapy, supplementation, or treatment. In one aspect, the method of administering the testosterone may include providing an oral dosage testosterone formulation as recited herein, and orally administering the formulation to a subject.

Various methods for making the testosterone oral dosage formulation of the present invention may be employed. However, as a general matter, such methods include forming a dispersion of testosterone in a molten polyethylene glycol carrier, cooling the dispersion into a solid mass, and dividing the mass into portions suitable for administration of a single testosterone dose. In some aspects, the testosterone may be uniformly dispersed in the carrier. In another aspect, such a method may additionally include extruding the molten dispersion while cooling to form an extrusion product. The extrusion product may then be cut into caplets or other suitably shaped forms for individual administration. In another embodiment, the solid mass, whether extruded or not during cooling, may be reduced to flakes, granules, powder, and then separated into single dosage amounts. The single dosage amounts may then be compacted into a solid state dosage form such as a tablet by pressing, molding, etc. In one aspect, the molding may be accomplished via injection molding. Alternatively, the single dosage amounts may be encapsulated with one or more encapsulating materials in order to form a capsule dosage.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION OF THE INVENTION

Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a drug” includes reference to one or more of such drugs, and reference to “the hormone” includes reference to one or more of such hormones.

As used herein, “formulation” and “composition” may be used interchangeably and refer to a combination of elements or materials that is presented together for a given purpose. Such terms are well known to those of ordinary skill in the art.

The terms “an effective amount” and “therapeutically effective amount may be used interchangeably. For example, an effective amount of testosterone is an amount that is sufficient to attain a specified therapeutic effect, or physiologic response or result that is brought about testosterone. The therapeutically effective blood level for bringing out such effect, response, or result may vary depending on the therapeutic benefit desired, as well as other variables such as the subject's age, weight, metabolism, presence of various enzymes, globulins, or other substances, and physiological conditions such as gastrointestinal motility, renal clearance, etc. Therapeutically effective blood levels may be achieved in one or more administrations, applications or dosage regimens. Determination of the dosing needed to achieve a therapeutically effective blood level for a given therapy or treatment is well within the ability of one of ordinary skill in the pharmaceutical arts. Typically, such blood levels may range from about 15 ng/dl to about 1200 ng/dl, (i.e. from about 300 ng/dl to about 1200 ng/dl for human males, and from about 15 ng/dl to about 80 ng/dl for human females.

As used herein, “carrier” refers to an ingredient or composition that contains, or is otherwise associated with, and aids in the preparation and/or delivery of a drug product which contains testosterone and is generally considered to be otherwise pharmacologically inactive.

As used herein, “a substantially solid polyethylene glycol carrier” refers to a carrier that comprises a polyethylene glycol that is substantially free of liquid components such as oils, liquid polymers, or other pharmaceutically acceptable fluid components or fluid excipients. It should be understood that such liquids or fluid excipients may be used for an auxiliary purpose such as to dissolve or disperse a given drug in the formulation, without destroying the solid nature of the dosage form.

As used herein, “oral dosage form” refers to a formulation that is ready for administration to a subject through the oral route of administration. Examples of known oral dosage forms, include without limitation, tablets, capsules, caplets, powders, pellets, and granules. Such formulations also include multilayered tablets wherein a given layer may represent a different drug. In some aspects, powders, pellets, and granules may be coated with a suitable polymer or a conventional coating material to achieve, for example, greater stability in the gastrointestinal tract, or to achieve the desired rate of release. Moreover, capsules containing a powder, pellets or granules may be further coated. Tablets and caplets may be scored to facilitate division of dosing. Alternatively, the dosage forms of the present invention may be unit dosage forms wherein the dosage form is intended to deliver one therapeutic dose per administration.

As used herein, “a subject” refers to a human, either male or female, or an animal (preferably a mammal) that are either in need of, or can benefit from, the administration of the testosterone.

As used herein, “injection molding” refers to a process for producing oral solid dosage forms wherein a predetermined amount of the formulation comprising a carrier and a pharmaceutical (along with optional adjuvants) are injected into a mold at a certain temperature and pressure and the mold is cooled and the oral solid dosage form is collected. Optionally, the mold can be packed with additional amount of the formulation material during the cooling cycle. In some aspects, the dosage form may be a tablet or a caplet.

As used herein, “blood level” is used interchangeably with terms such as blood concentration, plasma level, plasma concentration, serum level, serum concentration, serum blood level and serum blood concentration.

As used herein, “administration” refers to a method of delivering a formulation to a desired site. Specifically, oral administration refers to ingesting a drug by swallowing or chewing.

As used herein, “testosterone” refers to the steroid hormone having the IUPAC names (17 [symbol] )-17-Hydroxyandrost-4-en-3 -one, and [symbol] ⁴-androsten-1 [symbol] β-ol-3 -one. Testosterone is listed in the Merck Index, entry no. 9322, at page 1569, 12th ed., (1996), which is incorporated herein by reference. Testosterone may be obtained or prepared using the knowledge of one ordinarily skilled in the art from either a natural source, or synthetically using a process, such as those disclosed in U.S. Pat. No. 2,236,574 which is incorporated herein by reference. Further, a number of closely related androgenic compounds which are synthetically derivatized from testosterone are known to provide the same or a similar physiologic activity. Subsequently, the term “testosterone” is intended to include testosterone and its metabolites, salts, esters, derivatives, isomers, and prodrugs. Examples include, but are not limited to: acetate, enanthate, cypionate, isobutyrate, propionate, undecanoate, cyproterone acetate, danazol, finasteride, fluoxymesterone, methyltestosterone, nandrolone decanoate, nandrolone, phenpropionate, oxandrolone, oxymetholone, stanozolol, testolactone, 17\acute{α}-methylnortestosterone, norethandrolone, dihydrotestosterone, phenylacetate, buciclate, heptanoate, caprate, and isocaprate.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a range of 1 to 5 should be interpreted to include not only the explicitly recited limits of 1 and 5, but also to include individual values such as 2, 7, 3.6, 4.2, and sub-ranges such as 1-2.5, 1.8-3.2, 2.6-4.9, etc. This interpretation should apply regardless of the breadth of the range or the characteristic being described, and also applies to open-ended ranges reciting only one end point, such as “greater than 25,” or “less than 10”.

The Invention

The present invention provides oral dosage forms of testosterone and methods for the making and use thereof. The oral dosage form may be administered to both men and women to achieve desired testosterone blood levels in the subject receiving treatment. As such, the formulation may be used to treat a variety of diseases, disorders and conditions thought to be responsive to testosterone administration.

In one aspect, the testosterone dosage may be a solid oral dosage form of testosterone. Such an administration form will generally include a therapeutically effective amount of testosterone in a substantially solid polyethylene glycol carrier. The solid dosage form, upon oral administration, provides a therapeutically effective blood serum level of testosterone to a subject. The testosterone dosage forms of this invention may be prepared by injection molding techniques, or any other manufacturing method known to one skilled in the art.

The oral dosage forms of the present invention can be processed into an immediate release or a sustained release dosage form. Immediate release dosage forms may release the testosterone in a fairly short time, for example, within a few minutes to within a few hours. Sustained release dosage forms may release the testosterone over a period of several hours, for example, up to 24 hours or longer, if desired. In either case, the delivery can be controlled to be substantially at a certain predetermined rate over the period of delivery.

The oral dosage forms of the present invention can be processed into dosage forms such as tablets, capsules, caplets, powders, encapsulated pellets, encapsulated granules, or encapsulated powders. These dosage forms can be coated with a polymeric or other art-known coating materials to achieve, for example, greater stability on the shelf or in the gastrointestinal tract, or to achieve control over drug release. Such coating techniques and materials used therein are well-known in the art. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and hydroxypropylmethyl cellulose acetate succinate, among others, can be used to achieve enteric coating. Mixtures of waxes, shellac, zein, ethyl cellulose, acrylic resins, cellulose acetate, silicone elastomers can be used to achieve sustained release coating. See, for example, Remington, supra, Chapter 93, for other types of coatings, techniques and equipment.

Additionally, the tablet or caplet dosage form can be scored to facilitate easy break-off to adjust the dosage as needed. The tablets can also be multi-layered, each layer representing a different drug or a different concentration of the same drug. Alternatively, the dosage forms of the present invention can be prepared as unit dosage forms which are intended to deliver one therapeutically effective dose per administration.

General methods and equipment for preparing tablets, capsules, pellets, and powders are well-known in the art. See, Remington, supra, Chapters 91 and 92.

The present testosterone dosage forms constitute commonly used pharmaceutical excipients to form a stable natural testosterone product without the use of allergenic ingredients such as peanut oil. Additional advantages of these dosage forms include dosing flexibility, convenience, greater patient compliance in a clinical setting and the attendant benefits of improved clinical outcomes.

Testosterone

Testosterone administration has been shown to alleviate, ameliorate, or treat a wide variety of diseases, disorders, and conditions, including, without limitation: AIDS wasting syndrome, hypogonadism, somatopause, andropause, viropause, other androgen deficiencies in males, anemia, kidney disease, benign prostatic hyperplasia, acne, infertility, constipation, dry eyes, periodontal disease, diabetic retinopathy and other retinopathies, Lupus Erythematosis or other autoimmune diseases, decreased bone density or osteoporosis, heart disease, hyperlipidemias and angina. It has also been used to treat Addison's disease, hypopituitarism, and many be useful in accelerating growth in cases of pituitary dwarfism. It has also been shown to assist in the retention of calcium. Testosterone has been successfully used to treat various conditions in women subjects, such as menopause, endometriosis, postmenopausal vasomotor instability, and dysmenorrheal. Studies have shown that it may also be helpful in reducing some of the pain associated with breast cancer. Further, testosterone replacement therapy has been shown to provide positive health benefits to individuals deficient in testosterone, such as significantly increase muscle strength, mood, cognitive function and energy in men and women, increase insulin-like growth factor in serum and saliva, cause a temporary and reversible decrease in sperm count, and increase the penis size in prepubertal boys and hypogonadal adult men with micropenis. It may also be used to treat deficiencies in sexual desire, due to its effect of increasing libido in both males and females.

In one aspect of the present invention, the amount of testosterone present in the formulation is an amount that is sufficient to attain a target therapeutic result. Such amount can be readily determined by one of ordinary skill in the art given the nature of the particular condition to be treated or prevented. However, in one aspect, it is contemplated that the amount of testosterone will be at least 30% w/w of the solid oral dosage formulation. In another aspect, the amount of testosterone present in the formulation may be from about 5 mg to about 15 mg. In yet another aspect, the amount of testosterone in the formulation may be about 10 mg.

Substantially Solid Polyethylene Glycol Carrier

The solid oral dosage formulations of the present invention include a substantially solid polyethylene glycol carrier in combination with the testosterone. Any amount of carrier that is required in order to achieve a formulation with specifically desired characteristics may be used. However, in one aspect, the substantially solid polyethylene glycol carrier may be from about 30% w/w to about 80% w/w of the oral dosage formulation. In an additional aspect, the substantially solid polyethylene glycol carrier may be from about 50% w/w to about 80% w/w of the oral dosage formulation. In another aspect of the present invention, the substantially solid polyethylene glycol carrier may be from about 60% w/w to about 80% w/w of the oral dosage form. In yet another aspect, the substantially solid polyethylene glycol carrier may be about 70% w/w of the oral dosage form.

Polyethylene glycol is available in various grades under several trademarks including Carbowax® PEG 200, 300, 400, 540 Blend, 900, 1000, 1450, 3350, 4000, 4600, 8000 and compound 20M from Union Carbide Co., USA and Poly Glycols® E series from Dow chemical Co., USA. The various grades available under a given trademarks represent differences in molecular weight and viscosity.

In one aspect, the carrier is a mixture of polyethylene glycols having a molecular weight of from about 100 to about 20,000. In another aspect, the carrier is a mixture of polyethylene glycols having a molecular weight of from about 1000 to about 10,000. In some aspects, the polyethylene glycol is polyethylene glycol 1450, polyethylene glycol 3350 or polyethylene glycol 8000, or a mixture thereof.

It is to be understood that adding additional components to the polymers may be contemplated which is envisioned within the scope of the invention provided there is no deleterious effect on the overall composition and effective therapeutic provision of medication. Thus, in another aspect, the carrier may include a mixture of polyvinylpyrrolidones having a mean molecular weight ranging from 2,500 to 3,000,000 or more. There are many commercially available polyvinylpyrrolidone polymers suitable for the purposes of this invention. In another aspect, the carrier is a cellulose ether. Some exemplary cellulose ethers may include hydroxyalkyl cellulose (such as hydroxyethyl cellulose, hydroxypropyl cellulose, hydroxypropylmethyl cellulose, cellulose acetate trimellitate etc), and carboxyalkyl celluloses (such as carboxymethyl cellulose, carboxyethyl cellulose, etc) or a mixture thereof. In some aspects, the carrier may include adjuvants such as opacifiers, bulking agents, sweeteners, stabilizing agents, etc. Examples of opacifiers include titanium dioxide, Talc, calcium carbonate, behenic acid, and cetyl alcohol. Examples of bulking agents include starch, microcrystalline cellulose, calcium sulfate, calcium phosphate, and lactose. Examples of sweeteners include aspartame, saccharin, sodium cyclamate and Xylitol. Examples of stabilizing agents include alginic acid glycerylmonostearate, hydroxypropyl cellulose, magnesium, aluminum silicate, and propylene glycol.

The above carriers can be used to prepare dosage forms of this invention that can release the testosterone immediately, i.e., within a few minutes to within a few hours, or in a sustained manner, i.e., over a period of 24 hours or longer if desired. The dissolution rate of the testosterone dosage form can be influenced by including adjuvants such as surfactants to the dosage form. Examples of suitable surfactants include without limitation, sodium lauryl sulfate, glyceryl monooleate, sorbitan ester, docusate sodium, and cetrimide. The surfactant may constitute from about 0.1% to about 5% by weight of the dosage form. In one aspect, for example, a polyethylene glycol dosage form of the present invention as described herein may comprise about 2.5% by weight sodium lauryl sulfate to provide an immediate release dosage form.

In the case of sustained release dosage forms, additional solid carriers can be used, including, but not limited to, gums, acrylic resins or a mixture thereof.

Methods of Manufacture

In general, the methods of making an oral dosage testosterone formulation in accordance with the present invention include the steps of: 1) forming a dispersion of testosterone in a molten polyethylene glycol carrier; 2) cooling the dispersion into a solid mass; and 3) dividing the mass into portions suitable for administration of a single testosterone dose. Those of ordinary skill in the art will recognize the required temperatures for liquefying polyethylene glycol, depending on the specific molecular weight, or blend of molecular weights thereof In one aspect, the temperature will be sufficient to liquefy the polyethylene glycol without destabilizing or degrading the testosterone active agent. To be dispersed therein.

Once in a molten state, the polyethylene glycol is ready to receive the testosterone. Generally, the testosterone will be added in a solid crystalline form. However, in some aspect, the testosterone may be mixed with other solvents, adjuvants, etc. and then added to the molten polyethylene glycol in order to accommodate a particular formulation. The molten mixture may then be agitated, and in some aspect may be thoroughly mixed until the testosterone becomes uniformly, or substantially uniformly dispersed in the molten polyethylene glycol carrier.

After the testosterone and any additional adjuvants, excipients, or the like have been dispersed in the molten polyethylene glycol to form a drug and carrier mixture, the mixture is then cooled in order to allow the formulation to solidify. At this point, the formulation may be extruded or otherwise shaped in order to accommodate further processing, or it may be maintained in a bulk mass. Those of ordinary skill in the art will recognize the devices or apparatuses that can be suitably used for the purposes of extruding, or otherwise shaping the molten mixture as it cools.

Following the cooling, and optional extruding or shaping the now solid mass may be divided into portions, amounts, or quantities that are suitable for use as an individual dosage unit. A variety of mechanisms for dividing the solid mass may be used, the selection of which will depend largely on the final dosage formulation to be achieved. In one aspect, the solid mass may be in an extruded form which is then cut or otherwise separated into caplets. In another aspect, the solid mass may be reduced into granules, flakes, powder, or other sized or shaped particulates. Such materials may then be further processed into a specific dosage form as desired. For example, such materials may be molded or pressed into a typical solid state form, such as a tablet. Those of ordinary skill in the art will recognize a variety of mechanisms for accomplishing such forms. However, in one aspect, the oral solid dosage formulation of testosterone may be prepared with a technique known as injection molding. For a general description of this technique, see for example, U.S. Pat. Nos. 3,432,592, 4,801,460, 4,806,337, 5,004,601, and 5,082,655, and Cuff, G. and Raouf, F., Pharmaceutical Technology, 96-106 (1998), each of which are incorporated by reference herein in their entirety.

Briefly, polyethylene glycols are melted and testosterone is dispersed into the molten polyethylene glycol carrier. After the dispersion of the testosterone, a small amount of adjuvants such as titanium dioxide (as an opacifier) or pregelatinized starch (as a bulking agent) may be added to the mixture. The melted mixture is cooled down through a lentilling machine to form flakes of lentils. Lentils or even larger sized intermediate flakes can be prepared by melt granulation, lentilling and/or milling. The lentils and the intermediate flakes are passed through a screen of appropriate sieve size to form smaller pieces of lentils. These lentils are made into oral solid dosage forms such as tablets and caplets with techniques known to those skilled in the art, such as injection molding.

During injection molding, a mold is closed and clamped to prevent it from opening. The formulation comprising the pharmaceutical and its carrier is injected into the cavities of the mold through a nozzle. The amount of material that is injected into the mold is controlled by moving a screw to a predetermined distance inside the mold. The screw may be displaced to facilitate packing of additional material into the mold cavities to fill the void generated when the mold cools after the first injection. The various parameters of the injection and packing steps, such as packing time, packing pressure, injection rate, and injection pressure can be adjusted. The mold is cooled and the screw is returned to its pre-injection position. The mold is opened and the molded parts (in this case, the solid unit dosage forms) are ejected. See Cuff and Raouf, supra.

The molded dosage form can be scored with conventional techniques. Alternatively, the mold can be set such that scoring is accomplished through the molding process.

### EXAMPLES

The following examples are provided in order to promote a more clear understanding of certain aspects of the invention, and are in no way meant as a limitation thereon. Using the above-recited methodology various testosterone formulations are formed.

### Example 1

A composition for preparing Testosterone oral tablets constituting 10 mg of Testosterone per unit is provided as below in Table 1.

TABLE 1
10 mg Testosterone Formulation

| Component | mg/unit | % (by weight) |
|---|---|---|
| Testosterone (micronized or non- | 10.0 | 5.00 |
| micronized) | | |
| Polyethylene glycol 1000, NF | 15.00 | 7.5 |
| Polyethylene glycol 1450, NF | 31.00 | 15.50 |
| Polyethylene glycol 3350, NF | 76.00 | 38.00 |
| Polyethylene glycol 8000, NF | 16.00 | 8.00 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

### Example 2

Another composition for preparing Testosterone oral tablets constituting 10 mg of Testosterone per unit is provided as below in Table 2.

TABLE 2
10 mg Testosterone Formulation

| Component | mg/unit | % (by weight) |
|---|---|---|
| Testosterone (micronized or non- | 10.0 | 5.00 |
| micronized) | | |
| Polyethylene glycol 400, NF | 5.00 | 2.5 |
| Polyethylene glycol 1000, NF | 15.00 | 7.5 |
| Polyethylene glycol 1450, NF | 31.00 | 15.50 |
| Polyethylene glycol 3350, NF | 71.00 | 35.50 |
| Polyethylene glycol 8000, NF | 16.00 | 8.00 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

### Example 3

An additional composition for preparing Testosterone oral tablets constituting 10 mg of Testosterone per unit is provided as below in Table 3.

TABLE 3
10 mg Testosterone Formulation

| Component | mg/unit | % (by weight) |
|---|---|---|
| Testosterone (micronized or non- | 10.0 | 5.00 |
| micronized) | | |
| Polyethylene glycol 1450, NF | 46.00 | 23.00 |
| Polyethylene glycol 3350, NF | 76.00 | 38.00 |
| Polyethylene glycol 8000, NF | 16.00 | 8.00 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

### Example 4

A composition for preparing Testosterone oral tablets constituting 2.5 mg of Testosterone per unit is provided as below in Table 4.

TABLE 4
2.5 mg Testosterone Formulation

| Component | mg/unit | % (by weight) |
|---|---|---|
| Testosterone (micronized or non- | 2.5 | 1.25 |
| micronized) | | |
| Polyethylene glycol 1000, NF | 15.00 | 7.5 |
| Polyethylene glycol 1450, NF | 31.00 | 15.50 |
| Polyethylene glycol 3350, NF | 76.00 | 38.00 |
| Polyethylene glycol 8000, NF | 23.5 | 11.75 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

### Example 5

Another composition for preparing Testosterone oral tablets constituting 2.5 mg of Testosterone per unit is provided as below in Table 5.

TABLE 5
2.5 mg Testosterone Formulation

| Component | mg/unit | % (by weight) |
|---|---|---|
| Testosterone (micronized or non- | 2.5 | 1.25 |
| micronized) | | |
| Polyethylene glycol 400, NF | 5.00 | 2.5 |
| Polyethylene glycol 1000, NF | 15.00 | 7.5 |
| Polyethylene glycol 1450, NF | 31.00 | 15.50 |
| Polyethylene glycol 3350, NF | 71.00 | 35.50 |
| Polyethylene glycol 8000, NF | 23.50 | 11.75 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

### Example 6

An additional composition for preparing Testosterone oral tablets constituting 2.5 mg of Testosterone per unit is provided as below in Table.

TABLE 6
2.5 mg Testosterone Formulation

| Component | mg/unit | % (by weight) |
|---|---|---|
| Testosterone (micronized or non- | 2.5 | 1.25 |
| micronized) | | |
| Polyethylene glycol 1450, NF | 46.00 | 23.00 |
| Polyethylene glycol 3350, NF | 76.00 | 38.00 |
| Polyethylene glycol 8000, NF | 23.5 | 11.75 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

### Example 7

A composition for preparing Testosterone oral tablets constituting 30 mg of Testosterone per unit is provided as below in Table 7.

TABLE 7
10 mg Testosterone Formulation

| Component | mg/unit | % (by weight) |
|---|---|---|
| Testosterone (micronized or non- | 30.0 | 15.00 |
| micronized) | | |
| Polyethylene glycol 1000, NF | 15.00 | 7.5 |
| Polyethylene glycol 1450, NF | 21.00 | 11.50 |
| Polyethylene glycol 3350, NF | 66.00 | 33.00 |
| Polyethylene glycol 8000, NF | 16.00 | 8.00 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

### Example 8

Another composition for preparing Testosterone oral tablets constituting 30 mg of Testosterone per unit is provided as below in Table 8.

TABLE 8
30 mg Testosterone Formulation

| Component | mg/unit | % (by weight) |
|---|---|---|
| Testosterone (micronized or non- | 30.0 | 15.00 |
| micronized) | | |
| Polyethylene glycol 400, NF | 5.00 | 2.5 |
| Polyethylene glycol 1000, NF | 15.00 | 7.5 |
| Polyethylene glycol 1450, NF | 21.00 | 10.5 |
| Polyethylene glycol 3350, NF | 61.00 | 30.50 |
| Polyethylene glycol 8000, NF | 16.00 | 8.00 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

### Example 9

An additional composition for preparing Testosterone oral tablets constituting 10 mg of Testosterone per unit is provided as below in Table 9.

TABLE 9
10 mg Testosterone Formulation

| Component | mg/unit | % (by weight) |
|---|---|---|
| Testosterone (micronized or non- | 30.0 | 15.00 |
| micronized) | | |
| Polyethylene glycol 1450, NF | 36.00 | 18.00 |
| Polyethylene glycol 3350, NF | 66.00 | 33.00 |
| Polyethylene glycol 8000, NF | 16.00 | 8.00 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

By using the above-described formulation and the methodology described above, testosterone tablets/caplets are prepared.

The above-recited examples are expected to produce testosterone blood levels of from 15 ng/dl to about 1200 ng/dl depending on the particular dosing regimen established. Further, the onset of delivery can range from within a few minutes to several hours depending on the type of the dosage form (i.e. immediate release, or controlled release). Accordingly, the Tmax can vary from about 30 minutes to about 4 hours.

It is to be understood that the above-described compositions and methods are only illustrative of preferred aspects of the present invention. Numerous modifications and alternative arrangements may be devised by those skilled in the art without departing from the spirit and scope of the present invention and the appended claims are intended to cover such modifications and arrangements.

Thus, while the present invention has been described above with particularity and detail in connection with what is presently deemed to be the most practical and preferred aspects of the invention, it will be apparent to those of ordinary skill in the art that numerous modifications, including, but not limited to, variations in materials, temperature, function, order, amount, and manner of operation, assembly and use may be made without departing from the principles and concepts set forth herein.

1. A testosterone oral dosage formulation for administration to a subject comprising:
a substantially solid polyethylene glycol carrier which comprises from about 30% w/w to about 80% w/w of the formulation, having a molecular weight range of from about 100 to about 20,000 or a mixture thereof; and
a therapeutically effective amount of testosterone, or is pharmaceutically acceptable salts or esters, or a mixture thereof in the carrier ranging from about 2.5 mg to about 45 mg, said formulation providing a therapeutically effective testosterone serum level ranging from about 15 ng/dl to about 1200 ng/dl when administered to the subject.

2. The formulation of claim 1, wherein the amount of testosterone in the carrier is from about 5 mg to about 15 mg.

3. The formulation of claim 2, wherein the amount of testosterone is about 10 mg.

4. The formulation of claim 1, wherein the polyethylene glycol of said carrier has an average molecular weight of from about 400 to about 15,000.

5. The formulation of claim 4, wherein the polyethylene glycol of said carrier has an average molecular weight of from about 1,000 to about 10,000.

6. The formulation of claim 1, wherein the carrier comprises from about 50% w/w to about 80% w/w of the formulation.

7. The formulation of claim 6, wherein the carrier comprises from about 60% w/w to about 80% w/w of the formulation.

8. The formulation of claim 7, wherein the carrier comprises about 70% w/w of the formulation.

10. A testosterone oral dosage formulation for administration to a subject, comprising:
a substantially solid polyethylene glycol carrier having a molecular weight of from about 1,000 to about 10,000, said carrier comprising at least about 70% w/w of the formulation; and
from about 10 mg to about 15 mg of testosterone in the carrier.

11. A method of administering testosterone to a subject, comprising:
providing a testosterone formulation as recited in any one of claims 1-10; and
orally administering the formulation to the subject.

12. The method of claim 11, wherein the subject is a male.

13. The method of claim 11, wherein the subject is a female.

14. A method of treating or ameliorating a condition in a subject for which testosterone is effective comprising:
providing a testosterone formulation as recited in any one of claims 1-10; and
orally administering the formulation to the subject in a therapeutically effective regimen.

15. A method of making an oral dosage testosterone formulation, comprising:
forming a dispersion of testosterone in a molten polyethylene glycol carrier;
cooling the dispersion into a substantially solid mass; and
dividing the mass into portions suitable for administration of a single testosterone dose.

16. The method of claim 15, further comprising extruding the molten polyethylene glycol carrier during the step of cooling to form an extrusion product.

17. The method of claim 16, further comprising cutting the extrusion product into caplets.

18. The method of claim 15, wherein the step of dividing further comprises reducing the solid mass to flakes, granules, or powder and separating the flakes granules, or powder into a single dosage amount.

19. The method of claim 18, further comprising molding the single dosage unit into a solid state form.

20. The method of claim 19, wherein the step of molding is accomplished by injection molding.

21. The method of claim 19, wherein the step of molding is accomplished by pressing.

22. The method of claim 19, wherein the solid state form is a tablet.

23. The method of claim 18, further comprising encapsulating the single dosage amount with a capsule.

24. The method of claim 15, wherein the testosterone is uniformly dispersed in the molten polyethylene glycol carrier.
